(19) United States
(12) Patent Application Publication
(10) Pub. No.: US 2024/0350773 A1
(43) Pub. Date: Oct. 24, 2024

(54) CATHETER SYSTEM HAVING WINGS CONFIGURED TO COUPLE TOGETHER

(71) Applicant: Becton, Dickinson and Company, Franklin Lakes, NJ (US)

(72) Inventors: Mohankumar Natesan, Singapore (SG); Edwin Jeyaseelan A., Tamil Nadu (IN); Mukilan Balamurugan, Tamil Nadu (IN); Balaji Kannan, Bangalore (IN); Karthik Bhaskar, Coimbatore (IN)

(21) Appl. No.: 18/137,337

(22) Filed: Apr. 20, 2023

(51) Int. Cl. A61M 25/02; A61M 25/00; A61M 25/06

(52) U.S. Cl. CPC A61M 25/02; A61M 25/0097; A61M 25/0606

(57) ABSTRACT

A catheter system may include a catheter hub, which may include a distal end, a proximal end, and a lumen. The catheter system may include a first wing extending from the catheter hub, and a second wing opposite the first wing and extending from the catheter hub. The first wing and the second wing may be movable between a collapsed position and a folded position. The first wing may be configured to be coupled to the second wing to secure the first wing and the second wing in the folded position. The catheter system may include an end cap, and the first wing and the second wing may be coupled to the end cap in the folded position. The catheter hub may include a snap feature, and the first wing and the second wing may be coupled to the snap feature in the folded position.

### BACKGROUND

A common type of catheter system includes a peripheral intravenous catheter (“PIVC”) that is over-the-needle. As its name implies, the PIVC that is over-the-needle may be mounted over an introducer needle having a sharp distal tip. The catheter system may include a catheter adapter, the PIVC extending distally from the catheter adapter, and the introducer needle extending through the PIVC. The PIVC and the introducer needle may be assembled such that the distal tip of the introducer needle extends beyond the distal tip of the PIVC with the bevel of the needle facing up away from skin of the patient immediately prior to insertion into the skin. The PIVC and the introducer needle are generally inserted at a shallow angle through the skin into vasculature of the patient.

In order to verify proper placement of the introducer needle and/or the PIVC in the blood vessel, a clinician may confirm that there is flashback of blood in a flashback chamber of the catheter system or in a space between the introducer needle and the PIVC. Once placement of the introducer needle has been confirmed, the clinician may remove the introducer needle, leaving the PIVC in place for future blood withdrawal or fluid infusion.

In some instances, the catheter system, such as, for example, the BD NEOFLON™ Pro IV Cannula, available from Becton Dickinson and Company of Franklin Lakes, New Jersey, may include a housing that may fit over wings of the catheter system to aid gripping of the catheter system by the clinician. The housing, however, is somewhat bulky, costly to produce, and often is not used after insertion of the catheter system into the vasculature of the patient.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a catheter system that includes a first wing and a second wing configured to be coupled together in a folded position. In some embodiments, the catheter system may be disposed in the folded position during venipuncture and catheter advancement into a blood vessel of a patient. In some embodiments, the catheter system in the folded position may facilitate holding of the catheter system at the first wing and the second wing to insert the catheter into the blood vessel. In this way, the first wing and the second wing coupled together when the catheter system is in the folded position may form a firm push tab. In some embodiments, after the venipuncture and the catheter advancement are complete, the first wing and the second wing may be uncoupled from each other and brought to a collapsed or flat position for securement of the catheter system to skin of the patient and stabilization.

In some embodiments, the catheter system may include a catheter hub, which may include a distal end, a proximal end, and a lumen extending through the distal end and the proximal end. In some embodiments, the catheter system may include a first wing extending from the catheter hub and a second wing opposite the first wing and extending from the catheter hub. In some embodiments, the first wing and the second wing may be movable between a collapsed position and a folded position. In some embodiments, in the folded position, the first wing may be configured to be coupled to the second wing to secure the first wing and the second wing. In some embodiments, the catheter system may include a catheter extending from the distal end of the catheter hub. In some embodiments, the catheter system may include a needle hub coupled to the proximal end of the catheter hub, and an introducer needle extending distally from the needle hub through the catheter.

In a set of embodiments, the first wing may include a pin and the second wing may include a pocket. The pin may be configured to be secured within the pocket to couple the first wing to the second wing to secure the first wing and the second wing in the folded position. The first wing may include an inner fold line and an outer fold line. The second wing may include an inner fold line and an outer fold line. In the folded position, the first wing may be folded along the inner fold line of the first wing and the outer fold line of the first wing, and the second wing may be folded along the inner fold line of the second wing and the outer fold line of the second wing.

In another set of embodiments, the first wing may include a tongue and the second wing may include a groove. The tongue may extend in a distal direction in the collapsed position. The tongue may be disposed within the groove to couple the first wing to the second wing to secure the first wing and the second wing in the folded position. The first wing may include a first fold line and a second fold line. The second wing may include a first fold line and a second fold line. The first wing may be folded at the first fold line of the first wing and the second fold line of the first wing in the folded position. The second wing may be folded at the first fold line of the second wing and the second fold line of the second wing in the folded position.

In another set of embodiments, the first wing may include a hole, and the second wing may include a protrusion. The first wing may include a first fold line. The second wing may include a first fold line and a second fold line. The first wing may be folded at only the first fold line of the first wing in the folded position. The second wing may be folded at the first fold line of the second wing and the second fold line of the second wing in the folded position.

In another set of embodiments, the catheter system may include an end cap. The first wing and the second wing may be coupled to the end cap to secure the first wing and the second wing in the folded position. The end cap may include a first pin and a second pin opposing the first pin. The first wing may include a first socket and the second wing may include a second socket. The first pin may be disposed within the first socket and the second pin is disposed within the second socket.

In another set of embodiments, the catheter hub may include a snap feature. In the folded position, the first wing and the second wing may be configured to snap to the snap feature to secure the first wing and the second wing in the folded position. The snap feature may be disposed on a top of the catheter hub between the first wing and the second wing. The first wing may include a fold line and the second wing comprises a fold line. The first wing may be folded at the fold line of the first wing and the second wing is folded at the fold line of the second wing in the folded position.

In the collapsed position, the first wing may include a first portion extending outwardly from the catheter hub, and the second wing may include a first portion extending outwardly from the catheter hub. The first portion of the first wing and the first portion of the second wing may be configured to rest on skin of a patient. In the collapsed position, the first wing may include a second portion extending upwardly from the first portion of the first wing, and the second wing may include a second portion extending upwardly from the first portion of the second wing. The second portion of the first wing and the second portion of the second wing may be configured to snap to the snap feature in the folded position.

The snap feature may include a first ridge and a second ridge opposing the first ridge. The first wing may include a groove configured to snap to the first ridge and the second wing comprises a groove configured to snap to the second ridge. The first ridge and the second ridge may be aligned with a longitudinal axis of the catheter hub and disposed on a top of the catheter hub.

The first wing may include a first fold line and a second fold line. The second wing may include a first fold line and a second fold line. In the folded position, the first wing may be folded at the first fold line of the first wing and the second fold line of the first wing. In the folded position, the second wing may be folded at the first fold line of the second wing and the second fold line of the second wing.

In the collapsed position, the first wing may include a first portion extending outwardly from the catheter hub, and the second wing may include a first portion extending outwardly from the catheter hub. The first portion of the first wing and the first portion of the second wing may be configured to rest on skin of a patient. The first wing may include a second portion disposed perpendicular to the first portion of the first wing in the folded position. In some embodiments, the second wing may include a second portion disposed perpendicular to the first portion of the second wing in the folded position. The second portion of the first wing and the second portion of the second wing may be configured to snap to the snap feature in the folded position.

The first portion of the first wing and the second portion of the first wing may be divided by the first fold line of the first wing. The first portion of the second wing and the second portion of the second wing may be divided by the first fold line of the second wing. In the folded position, the first wing may be folded at the first fold line of the first wing and the second fold line of the first wing. In the folded position, the second wing may be folded at the first fold line of the second wing and the second fold line of the second wing.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of a prior art catheter system;

FIG. 1B is an upper perspective view of a housing of the prior art catheter system; an example vascular access system, according to some embodiments;

FIG. 2A is an upper perspective view of an example catheter system, illustrating an example catheter assembly of the catheter system in a folded position, according to some embodiments;

FIG. 2B is an upper perspective view of the catheter system of FIG. 2A, illustrating an example needle assembly removed from the catheter assembly, according to some embodiments;

FIG. 2C is an upper perspective view of the catheter assembly of FIG. 2A, illustrating an example pin and an example pocket and the catheter assembly in an example collapsed position, according to some embodiments;

FIG. 2D is an upper perspective view of the catheter assembly of FIG. 2A, illustrating the catheter assembly in the folded position, according to some embodiments;

FIG. 2E is a top view of the catheter assembly of FIG. 2A, illustrating the catheter assembly in the collapsed position, according to some embodiments;

FIG. 3A is an upper perspective view of an example catheter system, illustrating the catheter system in an example folded position, according to some embodiments;

FIG. 3B is an upper perspective view of an example catheter assembly of the catheter system of FIG. 3A, illustrating the catheter assembly in an example collapsed position, according to some embodiments;

FIG. 3C is an upper perspective view of the catheter assembly of the catheter system of FIG. 3A, illustrating the catheter assembly in the folded position, according to some embodiments;

FIG. 3D is a top view of the catheter assembly of the catheter system of FIG. 3A, illustrating the catheter assembly in the collapsed position, according to some embodiments;

FIG. 4A is a top view of an example catheter assembly in an example collapsed position, according to some embodiments;

FIG. 4B is an upper perspective view of the catheter assembly of FIG. 4A in the collapsed position, according to some embodiments;

FIG. 4C is an upper perspective view of the catheter assembly of FIG. 4A in an example folded position, according to some embodiments;

FIG. 5A is an upper perspective view of an example catheter assembly in an example folded position, according to some embodiments;

FIG. 5B is an upper perspective view of an example end cap that may be coupled to the catheter assembly of FIG. 5A, according to some embodiments;

FIG. 5C is an upper perspective view of an example catheter system that includes the catheter assembly of FIG. 5A in the folded position, according to some embodiments;

FIG. 5D is a top view of the catheter assembly of FIG. 5A in the collapsed position, according to some embodiments;

FIG. 5E is a side view of the catheter system of FIG. 5C, illustrating an example removable cover, according to some embodiments;

FIG. 5F is an exploded view of the catheter system of FIG. 5C, according to some embodiments;

FIG. 6A is a top view of an example catheter assembly, according to some embodiments;

FIG. 6B is a proximal end view of the catheter assembly of FIG. 6A, according to some embodiments;

FIG. 6C if a distal end view of the catheter assembly of FIG. 6A, according to some embodiments;

FIG. 6D is an enlarged distal end view of a portion of the catheter assembly of FIG. 6A, according to some embodiments;

FIG. 7A is a top view of an example catheter assembly, according to some embodiments;

FIG. 7B is an upper perspective view of the catheter assembly of FIG. 7B, according to some embodiments;

FIG. 7C is another upper perspective view of the catheter assembly of FIG. 7B, according to some embodiments;

FIG. 7D is another upper perspective view of the catheter assembly of FIG. 7B, according to some embodiments; and

FIG. 7E is another upper perspective view of the catheter assembly of FIG. 7B, showing example second portions removed for illustrative purposes, according to some embodiments.

### DESCRIPTION OF EMBODIMENTS

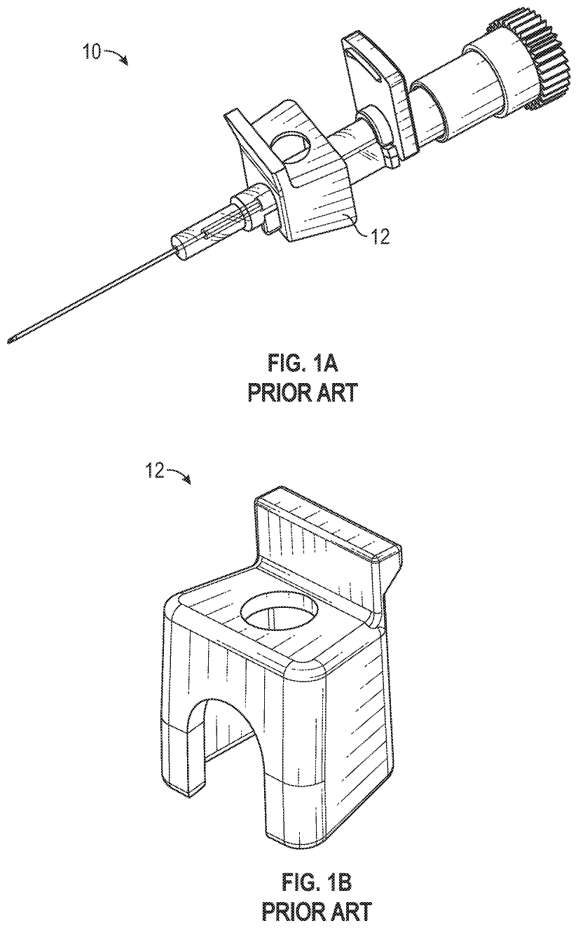

Referring now to FIGS. 1A-1B, a prior art catheter system 10 is illustrated, according to some embodiments. The prior art catheter system 10 may correspond to the BD NEOFLON™ Pro IV Cannula, available from Becton Dickinson and Company of Franklin Lakes, New Jersey. The prior art catheter system 10 includes a housing 12 that may fit over wings of the prior art catheter system 10 to aid gripping of the prior art catheter system 10 by a clinician during insertion of the prior art catheter system 10 into vasculature of a patient. The housing 12, however, is somewhat bulky, costly to produce, and often is not used after insertion of the prior art catheter system 10 into the vasculature of the patient. The wings and/or the housing 12 may be described further in U.S. patent application Ser. No. 16/989,243, filed Aug. 10, 2020, titled “CATHETER SYSTEM FOR PEDIATRIC SYSTEM.”

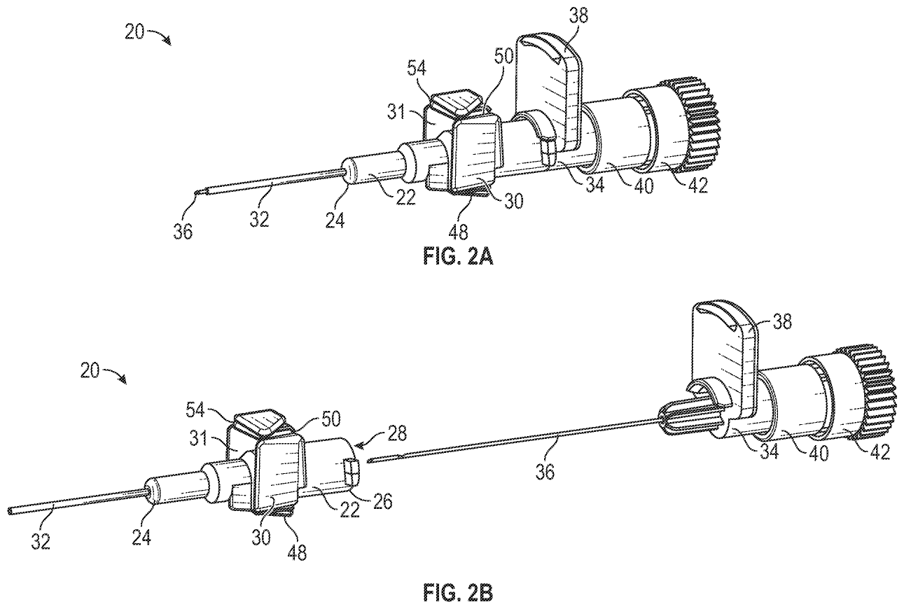

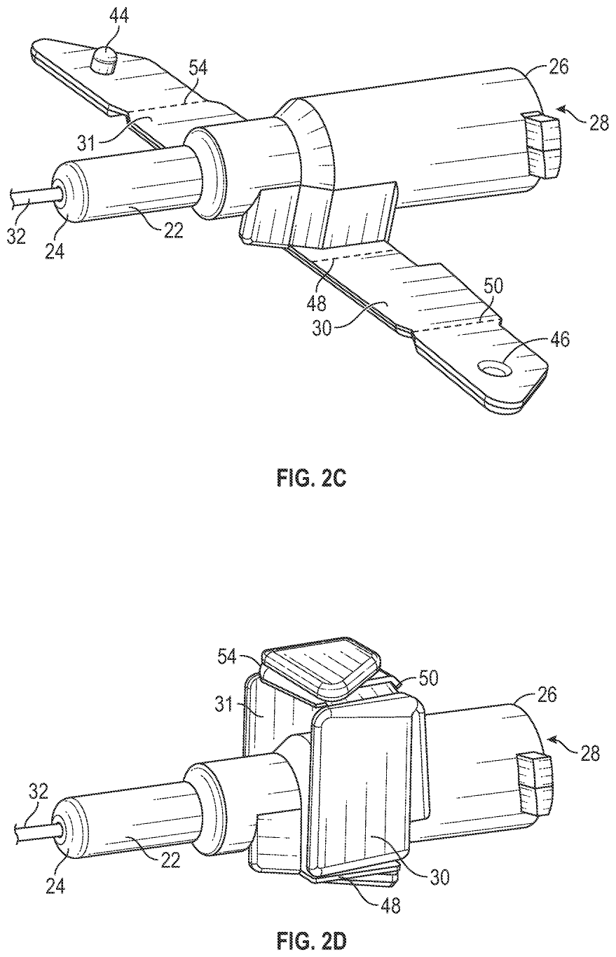

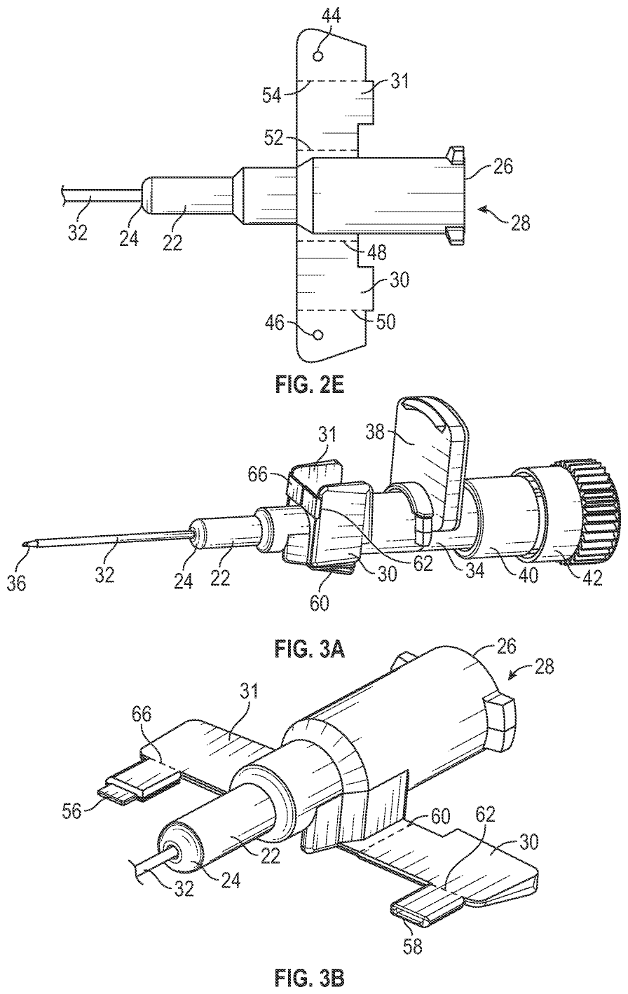

Referring now to FIGS. 2A-2E, in some embodiments, a catheter system 20 may include a catheter hub 22, which may include a distal end 24, a proximal end 26, and a lumen 28 extending through the distal end 24 and the proximal end 26. In some embodiments, the catheter system 20 may include a first wing 30 extending from the catheter hub 22 and a second wing 31 opposite the first wing 30 and extending from the catheter hub 22. In some embodiments, the first wing 30 and the second wing 31 may be movable between a collapsed position, illustrated, for example, in FIG. 2C, and a folded position, illustrated, for example, in FIGS. 2A, 2B, and 2D. In some embodiments, the first wing 30 may be configured to be coupled to the second wing 31 to secure the first wing 30 and the second wing 31 in the folded position.

In some embodiments, the catheter system 20 may include a catheter 32 extending from the distal end 24 of the catheter hub 22. In some embodiments, the catheter system 20 may include a needle hub 34 coupled to the proximal end 26 of the catheter hub 22, and an introducer needle 36 extending distally from the needle hub 34 through the catheter 32. In some embodiments, the needle hub 34 may include a push tab 38. In some embodiments, a vent plug 40 may be coupled to a proximal end of the needle hub 34. In some embodiments, the vent plug 40 may be configured to pass air and not blood to facilitate blood flashback, which may be visualized by the clinician in response to insertion of the introducer needle 36 into the vasculature of the patient. In some embodiments, an end cap 42 may be coupled to the vent plug 40 to prevent passing of the air until desired.

In some embodiments, the first wing 30 may include a pin 44 and the second wing 31 may include a pocket 46. In some embodiments, the pin 44 may be configured to be secured within the pocket 46 to couple the first wing 30 to the second wing 31 to secure the first wing 30 and the second wing 31 in the folded position. In some embodiments, the first wing 30 may include an inner fold line 48 and an outer fold line 50. In some embodiments, the second wing 31 may include an inner fold line 52 and an outer fold line 54.

In some embodiments, the inner fold line 48 and the outer fold line 50 of the first wing 30 may correspond to portions of the first wing 30 configured to fold. In some embodiments, the inner fold line 52 and the outer fold line 54 of the second wing 31 may correspond to portions of the second wing 31 configured to fold. In some embodiments, one or more of the inner fold lines 48, 52 and the outer fold lines 50, 54 may include a score or groove to facilitate folding. In some embodiments, the inner fold lines 48 and the outer fold line 50 may extend across a width of the first wing 30. In some embodiments, the inner fold line 52 and the outer fold line 54 may extend across a width of the second wing 31.

In some embodiments, the inner fold line 48 may be generally parallel to the outer fold line 50 of the first wing 30. In some embodiments, the inner fold line 52 may be generally parallel to the outer fold line 54 of the second wing 31. In some embodiments, one or more of the inner fold lines 48, 52 and the outer fold lines 50, 54 may be generally parallel to a longitudinal axis of the catheter hub 22. In some embodiments, in the folded position, the first wing 30 may be folded along the inner fold line 48 and the outer fold line 50, and the second wing 31 may be folded along the inner fold line 52 and the outer fold line 54. In some embodiments, ends of the first wing 30 and the second wing 31 may overlap in the folded position.

In some embodiments, the catheter system 20 may be disposed in the folded position during venipuncture and catheter advancement into a blood vessel of a patient. In some embodiments, the catheter system 20 in the folded position, with the pin 44 and the pocket 46 coupled together, may facilitate holding of the catheter system 20 at the first wing 30 and the second wing 31 to insert the catheter 32 into the blood vessel. In this way, the first wing 30 and the second wing 31 coupled together when the catheter system 20 is in the folded position may form a firm push tab. In some embodiments, after the venipuncture and the catheter advancement are complete, the first wing 30 and the second wing 31 may be uncoupled from each other and brought to a flat position for securement of the catheter system 20 to skin of the patient and stabilization.

As illustrated in FIG. 2E, in a collapsed position in which the first wing 30 and the second wing 31 are unfolded, the inner fold line 48 may be closer to the longitudinal axis of the catheter hub 22 than the outer fold line 50 and the inner fold line 52 may be closer to the longitudinal axis of the catheter hub 22 than the outer fold line 54.

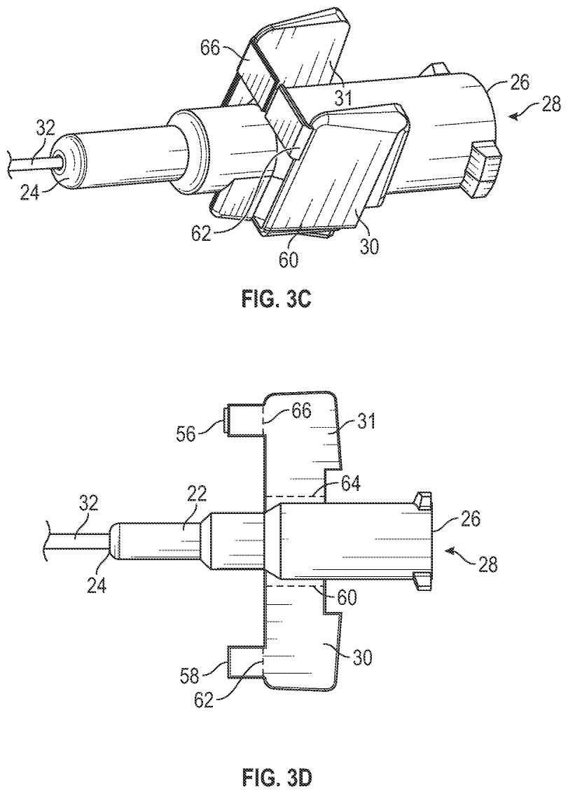

Referring now to FIGS. 3A-3C, in some embodiments, the first wing 30 may include a tongue 56 and the second wing 31 may include a groove 58. In some embodiments, the tongue 56 may extend in a distal direction in the collapsed position. In some embodiments, the tongue 56 may be disposed within the groove 58 to couple the first wing 30 to the second wing 31 to secure the first wing and the second wing in the folded position. In some embodiments, the first wing 30 may include a first fold line 60 and a second fold line 62. In some embodiments, the second wing 31 may include a first fold line 64 and a second fold line 66.

In some embodiments, the first wing 30 may be folded at the first fold line 60 of the first wing 30 and the second fold line 62 of the first wing 30 in the folded position. In some embodiments, the second wing 31 may be folded at the first fold line 64 of the second wing 31 and the second fold line 66 of the second wing 31 in the folded position. In some embodiments, the first fold line 60 of the first wing 30 may be generally perpendicular to the second fold line 62 of the first wing 30. In some embodiments, the first fold line 60 may be longer than the second fold line 62 of the first wing. In some embodiments, the first fold line 64 of the second wing 31 may be generally perpendicular to the second fold line 66 of the second wing 31. In some embodiments, the first fold line 64 of the second wing 31 may be longer than the second fold line 62 of the second wing 31. In some embodiments, one or more of the first fold line 60, the second fold line 62, the first fold line 64, and the second fold line 66 may include a score or groove to facilitate folding. In some embodiments, the first fold line 60 and the first fold line 64 may extend across a width of the first wing 30 and the second wing 31, respectively.

In some embodiments, the catheter system 20 may be disposed in the folded position during venipuncture and catheter advancement into a blood vessel of a patient. In some embodiments, the catheter system 20 in the folded position, with the tongue 56 and the groove 58 coupled together, may facilitate holding of the catheter system 20 at the first wing 30 and the second wing 31 to insert the catheter 32 into the blood vessel. In this way, the first wing 30 and the second wing 31 coupled together when the catheter system 20 is in the folded position may form a firm push tab. In some embodiments, after the venipuncture and the catheter advancement are complete, the first wing 30 and the second wing 31 may be uncoupled from each other and brought to a flat position for securement of the catheter system 20 to skin of the patient and stabilization.

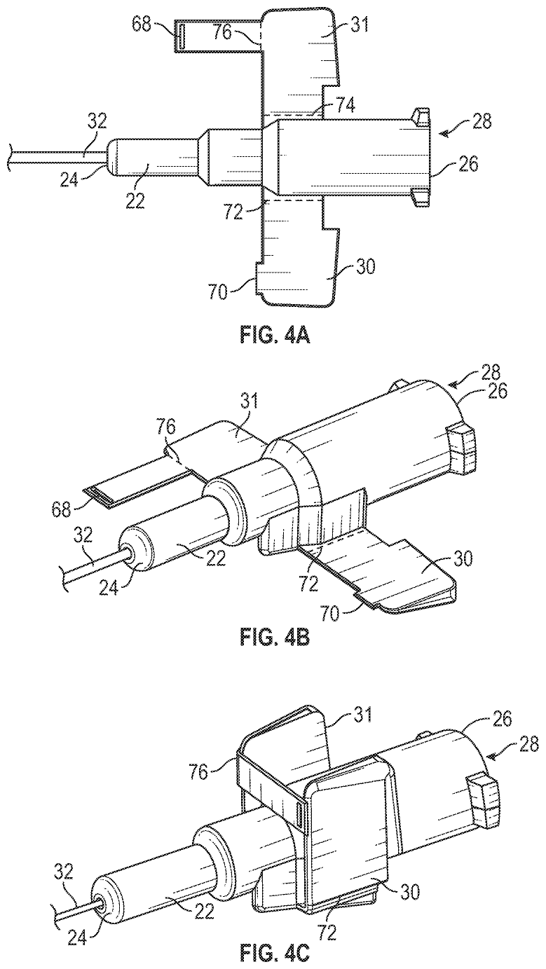

Referring now to FIGS. 4A-4C, in some embodiments, the first wing 30 may include a hole 68, and the second wing 31 may include a protrusion 70. In some embodiments, the first wing 30 may include a first fold line 72. In some embodiments, the second wing 31 may include a first fold line 74 and a second fold line 76. In some embodiments, the first wing 30 may be folded at only the first fold line of the first wing 30 in the folded position. In some embodiments, the second wing 31 may be folded at the first fold line 72 of the first wing 30 and the second fold line 76 of the second wing 31 in the folded position.

In some embodiments, one or more of the first fold line 72, the first fold line 74, and the second fold line 76 may include a score or groove to facilitate folding. In some embodiments, the first fold line 72 and the first fold line 74 may extend across a width of the first wing 30 and the second wing 31, respectively. In some embodiments, the second fold line 76 of the second wing 31 may be generally perpendicular to the first fold line 72 of the first wing 30 and the first fold line 74 of the second wing 31. In some embodiments, the first fold line 72 of the first wing 30 and the first fold line 74 may be generally parallel to the longitudinal axis of the catheter hub 22.

In some embodiments, the catheter system may be disposed in the folded position during venipuncture and catheter advancement into a blood vessel of a patient. In some embodiments, the catheter system in the folded position, with the hole 68 and the protrusion 70 coupled together, may facilitate holding of the catheter system at the first wing 30 and the second wing 31 to insert the catheter 32 into the blood vessel. In this way, the first wing 30 and the second wing 31 coupled together when the catheter system is in the folded position may form a firm push tab. In some embodiments, after the venipuncture and the catheter advancement are complete, the first wing 30 and the second wing 31 may be uncoupled from each other and brought to a flat position for securement of the catheter system to skin of the patient and stabilization. The catheter assembly of FIGS. 4A-4C may include one or more of the introducer needle 36, the needle hub 34, the vent plug 40, and the end cap 42 of FIGS. 2-3, for example.

Referring now to 5A-5F, in some embodiments, the catheter system 20 may include an end cap 78. In some embodiments, the first wing 30 and the second wing 31 may be coupled to the end cap 78 to secure the first wing 30 and the second wing 31 in the folded position. In some embodiments, the end cap 78 may include a first pin 80 and a second pin 82 opposing the first pin 80. In some embodiments, the first wing 30 may include a first socket 84 and the second wing 31 may include a second socket 86. In some embodiments, the first pin 80 may be disposed within the first socket 84 and the second pin 82 may be disposed within the second socket 86.

In some embodiments, the first wing 30 may include a first fold line 88, and the second wing 31 may include a first fold line 90. In some embodiments, the first wing 30 may include only the first fold line 88, and the second wing 31 may include only the first fold line 90. In some embodiments, the first fold line 88 and the second fold line 90 may be generally parallel to the longitudinal axis of the catheter hub 22.

In some embodiments, the first fold line 88 and the first fold line 90 of the first wing 30 and the second wing 31, respectively, may include a score or groove to facilitate folding. In some embodiments, the first fold line 88 of the first wing 30 and the first fold line 90 of the second wing 31 may extend across a width of the first wing 30 and the second wing 31, respectively. In some embodiments, the first fold line 88 and the first fold line 90 of the first wing 30 and the second wing 31, respectively, may be generally parallel to the longitudinal axis of the catheter hub 22.

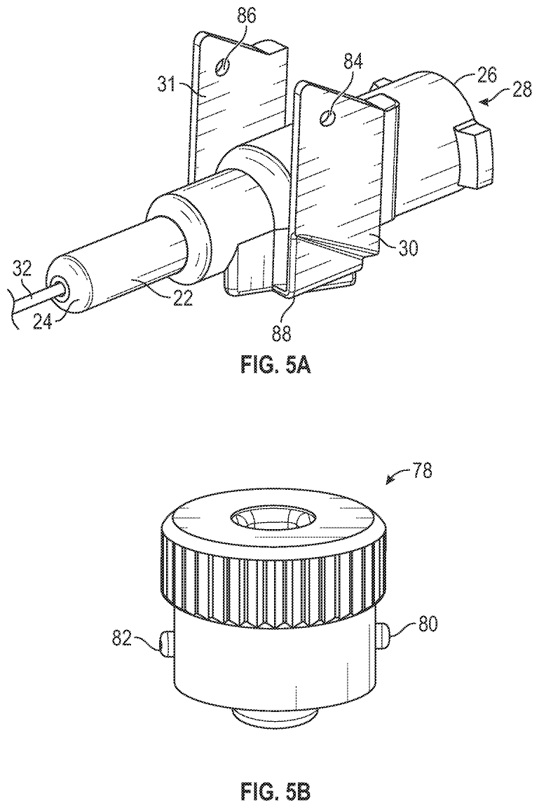

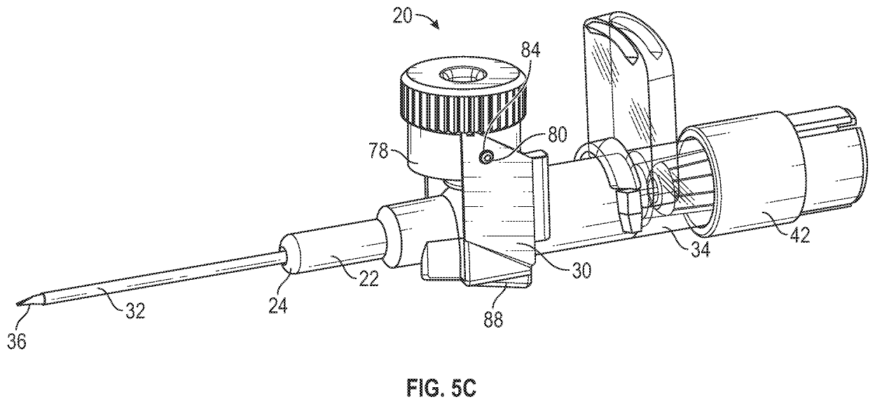

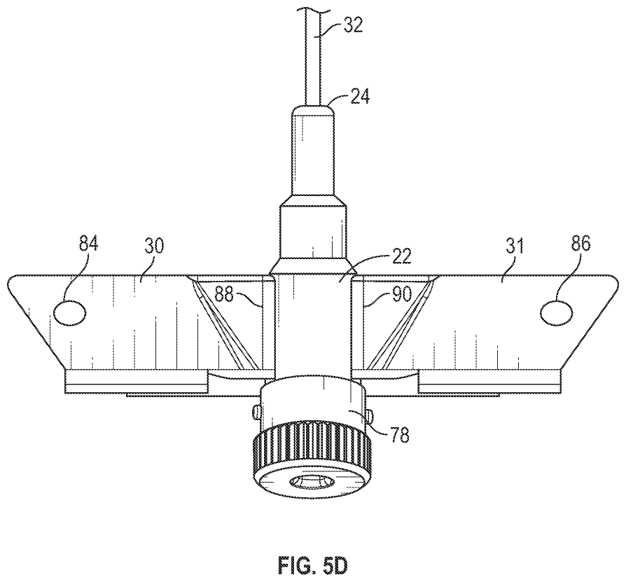

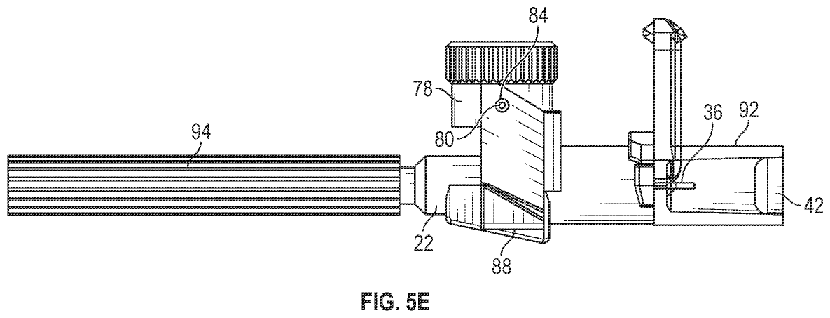

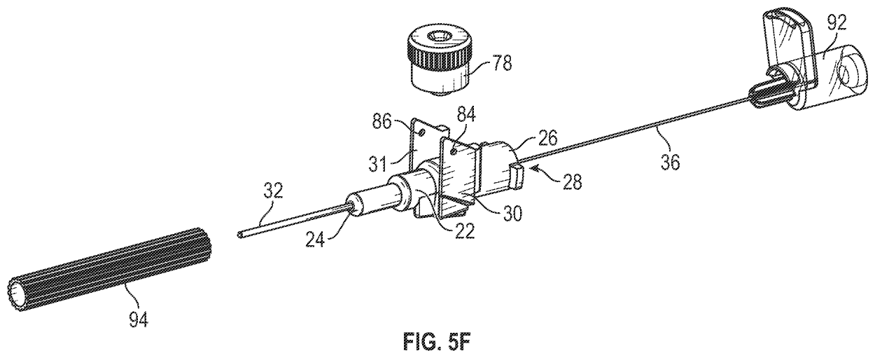

In some embodiments, the catheter system 20 may be disposed in the folded position during venipuncture and catheter advancement into a blood vessel of a patient. In some embodiments, the catheter system 20 in the folded position, with the first pin 80 and the second pin 82 coupled to the first socket 84 and the second socket 86, respectively, may facilitate holding of the catheter system 20 at the first wing 30 and the second wing 31 to insert the catheter 32 into the blood vessel. In this way, the first wing 30 and the second wing 31 coupled to the end cap 78 when the catheter system 20 is in the folded position may form a firm push tab. In some embodiments, after the venipuncture and the catheter advancement are complete, the first wing 30 and the second wing 31 may be uncoupled from the end cap 78 and brought to a flat position for securement of the catheter system 20 to skin of the patient and stabilization. In some embodiments, after the needle assembly is removed, the end cap 78 may be coupled to the proximal end 26 of the catheter hub 22, as illustrated in FIG. 5D, for example.

In some embodiments, the vent plug 40 that is a separate, removable piece may be eliminated. As illustrated in FIG. 5E-5F, for example, a needle hub 92 may include a venting element 93 within a proximal end of the needle hub 92. The venting element 93 may allow air to pass but not liquid. In some embodiments, the venting element 93 may include a paper cork, cellulose fiber with pores, or another suitable material to provide a venting effect. In some embodiments, a removable cover 94 may be provided over top of the catheter 32 and a sharp distal tip of the introducer needle 36. The needle hub 92 may be used with a particular catheter assembly of any of the other Figures in the present application. In some embodiments, the needle hub 92 may facilitate detection of venipuncture by allowing the clinician to visualize blood within the needle hub 92 distal to the venting element 93.

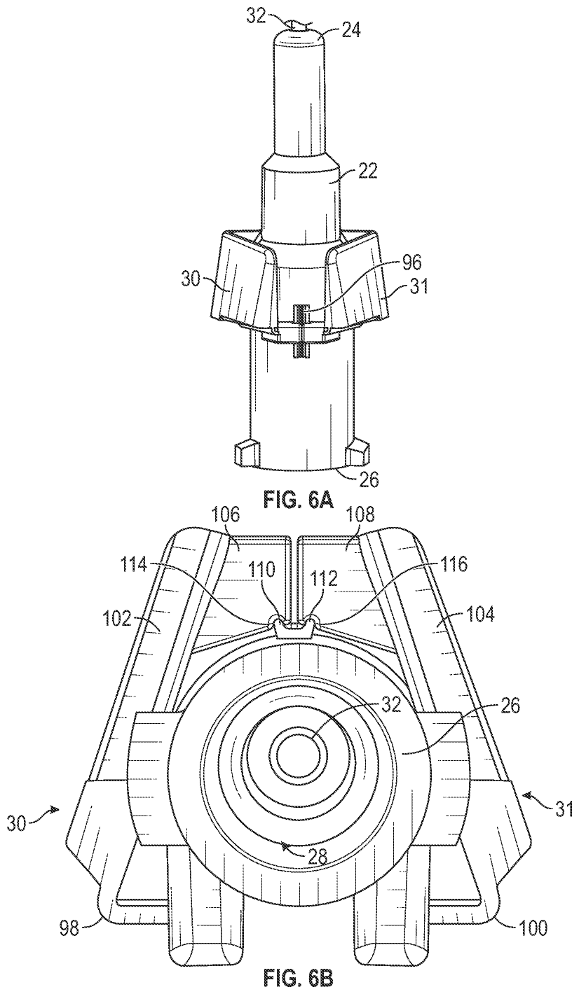

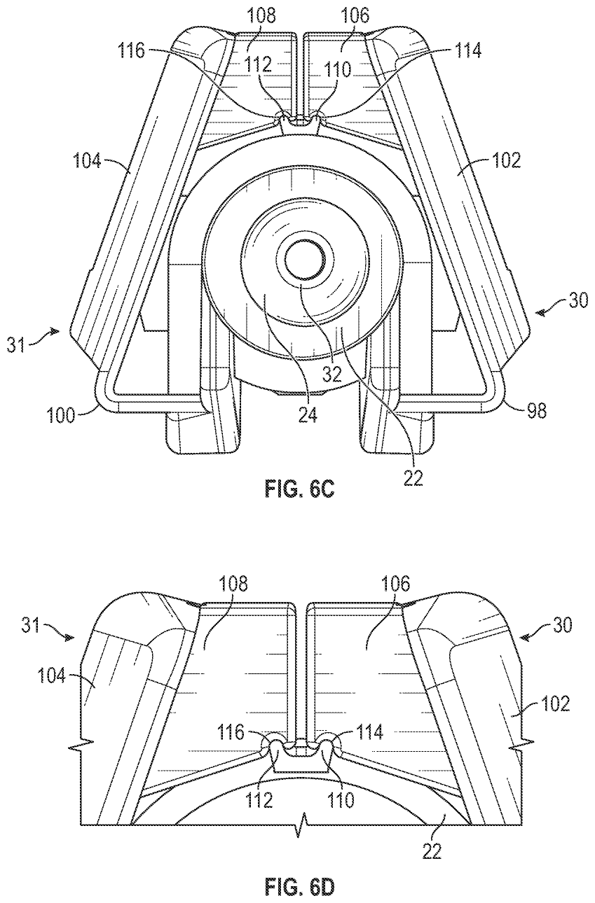

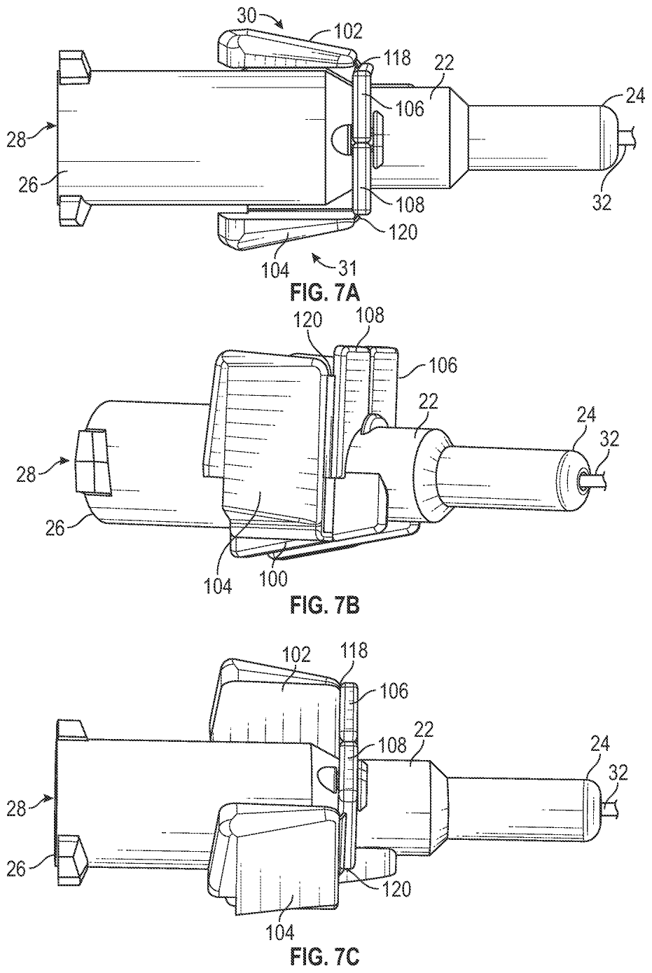

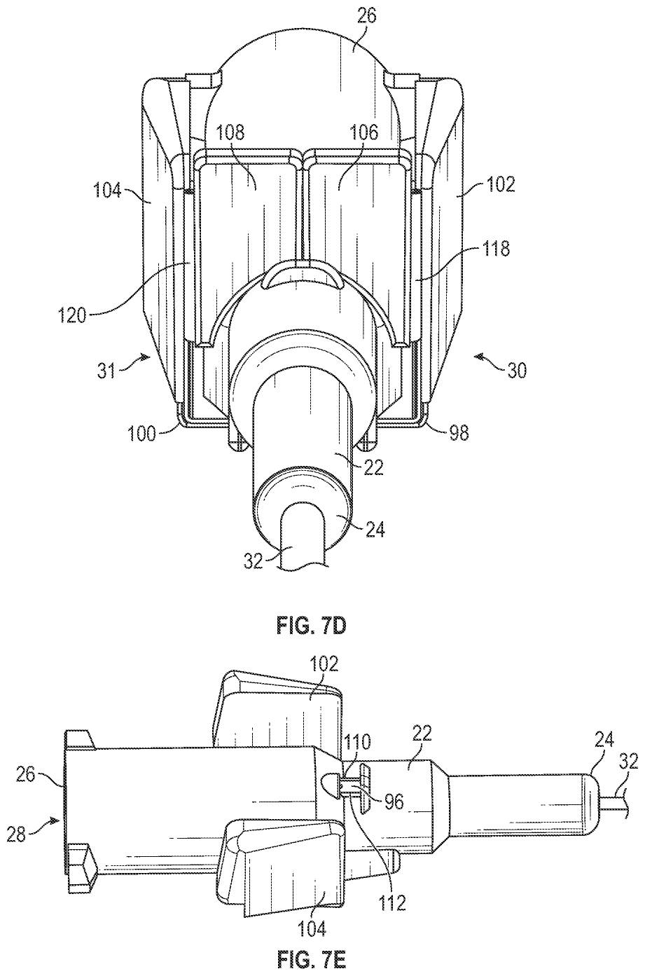

Referring now to FIGS. 6A-7E, in some embodiments, the catheter hub 22 may include a snap feature 96. In some embodiments, in the folded position, the first wing 30 and the second wing 31 may be configured to snap to the snap feature 96 to secure the first wing 30 and the second wing 31 in the folded position. In some embodiments, the snap feature 96 may be disposed on a top of the catheter hub 22 between the first wing 30 and the second wing 31.

Referring now to FIGS. 6A-6D, in some embodiments, the first wing 30 may include a first fold line 98 and the second wing 31 may include a first fold line 100. In some embodiments, the first wing 30 and the second wing 31 may include no more fold lines than the first fold line 98 and the first fold line 100, respectively. In some embodiments, the first wing 30 may be folded at the first fold line 98 of the first wing 30 and the second wing 31 may be folded at the first fold line 100 of the second wing 31 in the folded position.

In some embodiments, the first fold line 98 and the first fold line 100 of the first wing 30 and the second wing 31, respectively, may include a score or groove to facilitate folding. In some embodiments, the first fold line 98 of the first wing 30 and the first fold line 100 of the second wing 31 may extend across a width of the first wing 30 and the second wing 31, respectively. In some embodiments, the fold line 98 and the first fold line 100 of the first wing 30 and the second wing 31, respectively, may be generally parallel to the longitudinal axis of the catheter hub 22.

In some embodiments, in the collapsed position, the first wing 30 may include a first portion 102 extending outwardly from the catheter hub 22, and the second wing 31 may include a first portion 104 extending outwardly from the catheter hub 22. In some embodiments, the first portion 102 of the first wing 30 and the first portion 104 of the second wing 31 may be configured to rest on skin of a patient. In some embodiments, in the collapsed position, the first wing 30 may include a second portion 106 extending upwardly from the first portion 102 of the first wing 30, and the second wing 31 may include a second portion 108 extending upwardly from the first portion 104 of the second wing 31. In some embodiments, the second portion 106 of the first wing 30 and the second portion 108 of the second wing 31 may be configured to snap to the snap feature in the folded position.

In some embodiments, the snap feature 96 may include a first ridge 110 and a second ridge 112 opposing the first ridge 110. In some embodiments, the first wing 30 may include a groove 114 configured to snap to the first ridge 110 and the second wing 31 may include a groove 116 configured to snap to the second ridge 112. In some embodiments, the first ridge 110 and the second ridge 112 may be aligned with the longitudinal axis of the catheter hub 22 and/or disposed on a top of the catheter hub 22.

Referring now to FIGS. 7A-7E, in some embodiments, the first wing 30 may include the first fold line 98 and a second fold line 118. In some embodiments, the second wing 31 may include the first fold line 100 and a second fold line 120. In some embodiments, in the folded position, the first wing 30 may be folded at the first fold line 98 of the first wing 30 and the second fold line 118 of the first wing 30. In some embodiments, in the folded position, the second wing 31 may be folded at the first fold line 100 of the second wing 31 and the second fold line 118 of the second wing 31.

In some embodiments, in the collapsed position, the first wing 30 may include a first portion 122 extending outwardly from the catheter hub 22, and the second wing 31 may include a first portion 124 extending outwardly from the catheter hub 22. In some embodiments, the first portion 122 of the first wing 30 and the first portion 124 of the second wing 31 may be configured to rest on skin of the patient. In some embodiments, the first wing 30 may include a second portion 126 disposed perpendicular to the first portion 122 of the first wing 30 in the folded position. In some embodiments, the second wing 31 may include a second portion 128 disposed perpendicular to the first portion 124 of the second wing 31 in the folded position. In some embodiments, the second portion 126 of the first wing 30 and the second portion 128 of the second wing 31 may be configured to snap to the snap feature 96 in the folded position. In these embodiments, the second portion 126 of the first wing 30 and the second portion 128 of the second wing 31 may each include a groove, similar to groove 114 and groove 116 of FIG. 6.

In some embodiments, the first portion 122 of the first wing 30 and the second portion 126 of the first wing 30 may be divided by the first fold line 98 of the first wing 30. In some embodiments, the first portion 124 of the second wing 31 and the second portion 128 of the second wing 31 may be divided by the first fold line 100 of the second wing 31. In some embodiments, in the folded position, the first wing 30 may be folded at the first fold line 98 of the first wing 30 and the second fold line 118 of the first wing 30. In some embodiments, in the folded position, the second wing 31 may be folded at the first fold line 100 of the second wing 31 and the second fold line 120 of the second wing 31.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter hub, comprising a distal end, a proximal end, and a lumen extending through the distal end and the proximal end;
a first wing extending from the catheter hub;
a second wing opposite the first wing and extending from the catheter hub, wherein the first wing and the second wing are movable between a collapsed position and a folded position, wherein the first wing is configured to be coupled to the second wing to secure the first wing and the second wing in the folded position; and
a catheter extending from the distal end of the catheter hub.

2. The catheter system of claim 1, wherein the first wing comprises a pin and the second wing comprises a pocket, wherein the pin configured to be secured within the pocket to couple the first wing to the second wing to secure the first wing and the second wing in the folded position.

3. The catheter system of claim 2, wherein the first wing comprises an inner fold line and an outer fold line, wherein the second wing comprises an inner fold line and an outer fold line, wherein in the folded position, the first wing is folded along the inner fold line of the first wing and the outer fold line of the first wing, wherein in the folded position, the second wing is folded along the inner fold line of the second wing and the outer fold line of the second wing.

4. The catheter system of claim 1, wherein the first wing comprises a tongue and the second wing comprises a groove, wherein the tongue extends in a distal direction in the collapsed position, wherein the tongue is disposed within the groove to couple the first wing to the second wing to secure the first wing and the second wing in the folded position.

5. The catheter system of claim 3, wherein the first wing comprises a first fold line and a second fold line, wherein the second wing comprises a first fold line and a second fold line, wherein the first wing is folded at the first fold line of the first wing and the second fold line of the first wing in the folded position, wherein the second wing is folded at the first fold line of the second wing and the second fold line of the second wing in the folded position.

6. The catheter system of claim 1, wherein first wing comprises a hole, wherein the second wing comprises a protrusion, wherein the first wing comprises a first fold, wherein the second wing comprises a first fold line and a second fold line, wherein the second wing is folded at the first fold line of the second wing and the second fold line of the second wing in the folded position, wherein the first wing is folded at only the first fold line of the first wing in the folded position.

7. The catheter system of claim 1, further comprising: a needle hub coupled to the proximal end of the catheter hub; and an introducer needle extending distally from the needle hub through the catheter.

8. A catheter system, comprising:
a catheter hub, comprising a distal end, a proximal end, and a lumen extending through the distal end and the proximal end;
an end cap;
a first wing extending from the catheter hub;
a second wing opposite the first wing and extending from the catheter hub, wherein the first wing and the second wing are movable between a collapsed position and a folded position, wherein in the folded position, the first wing and the second wing are coupled to the end cap to secure the first wing and the second wing in the folded position; and
a catheter extending from the distal end of the catheter hub.

9. The catheter system of claim 8, wherein the end cap comprises a first pin and a second pin opposing the first pin, wherein the first wing comprises a first socket and the second wing comprises a second socket, wherein the first pin is disposed within the first socket and the second pin is disposed within the second socket.

10. The catheter system of claim 8, further comprising: a needle hub coupled to the proximal end of the catheter hub; and an introducer needle extending distally from the needle hub through the catheter.

11. A catheter system, comprising:
a catheter hub, comprising a distal end, a proximal end, a lumen extending through the distal end and the proximal end, and a snap feature;
a first wing extending from the catheter hub;
a second wing opposite the first wing and extending from the catheter hub, wherein the first wing and the second wing are movable between a collapsed position and a folded position, wherein in the folded position, the first wing and the second wing are configured to snap to the snap feature to secure the first wing and the second wing in the folded position; and
a catheter extending from the distal end of the catheter hub.

12. The catheter system of claim 11, wherein the snap feature is disposed on a top of the catheter hub between the first wing and the second wing.

13. The catheter system of claim 11, wherein the first wing comprises a fold line and the second wing comprises a fold line, wherein the first wing is folded at the fold line of the first wing and the second wing is folded at the fold line of the second wing in the folded position.

14. The catheter system of claim 13, wherein in the collapsed position, the first wing comprises a first portion extending outwardly from the catheter hub and the second wing comprises a first portion extending outwardly from the catheter hub, wherein the first portion of the first wing and the first portion of the second wing are configured to rest on skin of a patient, wherein in the collapsed position, the first wing further comprises a second portion extending upwardly from the first portion of the first wing and the second wing comprises a second portion extending upwardly from the first portion of the second wing, wherein the second portion of the first wing and the second portion of the second wing are configured to snap to the snap feature in the folded position.

15. The catheter system of claim 11, wherein the snap feature comprises a first ridge and a second ridge opposing the first ridge, wherein the first wing comprises a groove configured to snap to the first ridge and the second wing comprises a groove configured to snap to the second ridge.

16. The catheter system of claim 15, wherein the first ridge and the second ridge are aligned with a longitudinal axis of the catheter hub and disposed on a top of the catheter hub.

17. The catheter system of claim 11, wherein the first wing comprises a first fold line and a second fold line, wherein the second wing comprises a first fold line and a second fold line, wherein the first wing is folded at the first fold line of the first wing and the second fold line of the first wing in the folded position, wherein the second wing is folded at the first fold line of the second wing and the second fold line of the second wing in the folded position.

18. The catheter system of claim 17, wherein in the collapsed position, the first wing comprises a first portion extending outwardly from the catheter hub and the second wing comprises a first portion extending outwardly from the catheter hub, wherein the first portion of the first wing and the first portion of the second wing are configured to rest on skin of a patient, wherein the first wing further comprises a second portion disposed perpendicular to the first portion of the first wing in the folded position, wherein the second wing further comprises a second portion disposed perpendicular to the first portion of the second wing in the folded position, wherein the second portion of the first wing and the second portion of the second wing are configured to snap to the snap feature in the folded position.

19. The catheter system of claim 18, wherein the first portion of the first wing and the second portion of the first wing are divided by the first fold line of the first wing, wherein the first portion of the second wing and the second portion of the second wing are divided by the first fold line of the second wing, wherein in the folded position, the first wing is folded at the first fold line of the first wing and the second fold line of the first wing, wherein in the folded position, the second wing is folded at the first fold line of the second wing and the second fold line of the second wing.

20. The catheter system of claim 11, further comprising: a needle hub coupled to the proximal end of the catheter hub; and an introducer needle extending distally from the needle hub through the catheter.
